(19) United States
(12) Patent Application Publication
(10) Pub. No.: US 2026/0347940 A1
(43) Pub. Date: Nov. 5, 2026

(54) COMPOSITIONS AND METHODS OF DETERMINING LUPUS NEPHRITIS CLASS

(71) Applicant: YALE UNIVERSITY, New Haven, CT (US)

(72) Inventors: Mathieu BAKHOUM, New Haven, CT (US); Christine BAKHOUM, New Haven, CT (US)

(21) Appl. No.: 19/486,954

(22) PCT Filed: May 23, 2024
(86) PCT No.: PCT/US2024/030694

§ 371 Date: Nov. 24, 2025

Related U.S. Application Data
(60) Provisional application No. 63/503,947, filed on May 23, 2023.

(51) Int. Cl. C12Q 1/6883

(52) U.S. Cl. CPC C12Q 1/6883; C12Q 2600/106; C12Q 2600/112; C12Q 2600/154

(57) ABSTRACT

It has been determined that differential methylation at CpG loci can be used to determine the severity of lupus nephritis (LN). Thus, differentially methylated CpG loci (also referred to herein as biomarkers), as well as methods of detecting methylation at the biomarkers are provided, as are their use to determine the severity of LN and inform treatment. In particular, the differential methylation is useful in distinguishing patients with class II LN from patients suffering from LN of classes III-VI and thereby determining whether the patient should be given immunosuppressants.

### CROSS-REFERENCE TO RELATED APPLICATIONS

This application claims the benefit of and priority to U.S. Ser. No. 63/503,947, filed May 23, 2023, which is incorporated by reference herein in its entirety.

### FIELD OF THE INVENTION

The field of the invention is generally directed to lupus nephritis, means of determining the severity (i.e., class) of the disease, and using the determined severity to inform patient management decisions such as treatment.

### BACKGROUND OF THE INVENTION

Approximately 60% of patients with systemic lupus erythematosus (SLE) will eventually develop kidney involvement, or lupus nephritis. The pathogenesis of LN is complex and involves both the innate and adaptive immune system, various cytokines and tissue, and immune cells. Intra-renal inflammation is maintained via local cytokine and chemokine production and by cells of the innate immune system, such as neutrophils, that are attracted into the glomerulus and interstitium. Targeting local release of pro-inflammatory cytokines by blocking individual cytokines, may enhance treatment efficacy in autoimmunity without increasing systemic immunosuppression. (Allam (2008) Curr Opin Rheumatol; 20 (5): 538-44; Yu et al. (2017) Nat Rev Nephrol; 13 (8): 483-95). A kidney biopsy is routinely performed in patients with lupus nephritis, as the histopathological classification of disease on biopsy guides immunosuppressive treatment. The main risk of kidney biopsies is bleeding, and patients with lupus often have higher risk for bleeding as they can be anemic or thrombocytopenic.

Thus, there is a need to new and less invasive ways of categorizing the severity of lupus nephritis.

It is object of the invention to provide biomarkers for the severity of lupus nephritis, and methods of detection related thereto.

It is further object of the invention to provide methods of categorizing the severity of lupus nephritis.

It is further object of the invention to provide treatment of subjects with lupus nephritis guided by a determination of disease severity.

### SUMMARY OF THE INVENTION

It has been determined that differential methylation at CpG loci can be used to determine the severity (i.e., class or group of classes) of lupus nephritis. Thus, differentially methylated CpG loci (also referred to herein as biomarkers), as well as methods of detecting methylation at the biomarkers are provided, as are their use to determine the severity of disease and inform treatment.

Methods for determining lupus nephritis class are provided. The methods typically include detecting methylation at one or more differentially methylated CpG loci and comparing the level of methylation to one or more controls or references. The control(s) or reference(s) can be known methylation values for the same CpG loci in subjects previously categorized as being Class II or less, or Class III or higher. In some forms, the control(s) or reference(s) are known methylation values for the same CpG loci in subjects previously diagnosed as being Class II, Class III, Class IV, or Class V.

CpG loci that are differentially methylated in different classes of lupus nephritis are also provided. Exemplary loci include cg09713586_BC21, cg23059826_TC21, cg13350270_BC21, cg27416966_BC21, cg05108591_BC21, cg04040637_BC21, cg22350135_BC21, cg26419274_TC21, cg12477512_BC21, cg17310601_BC21, cg19741112_TC21, cg11661493_TC21, cg10419144_TC21, cg25901241_BC21, cg08637101_BC21, cg12062757_BC21, cg16552271_TC21, cg00315056_TC21, cg03444505_BC21, cg06093355_BC21, cg04378177_TC21, cg16538390_BC21, cg15183647_BC21, cg04994762_BC21, cg13745646_BC21, cg18832251_BC21, cg20695119_BC21, cg02884024_BC11, cg06441554_BC21, cg04806722_BC21, cg22249302_BC21, cg07044006_BC21, cg05764585_TC21, cg16284456_TC21, cg14061946_BC21, cg09463032_TC11, cg02748908_BC11, cg24057166_TC21, cg13283765_BC21, cg13561626_BC21, cg07642801_BC21, cg11130441_TC21, cg06419236_TC21, cg09398527_TC21, cg10085612_BC21, cg06237697_TC11, cg17550329_BC21, cg26615813_TC11, cg11317269_TC21, and cg21288300_BC21. For example, Cg05108591_BC21 is hypomethylated in Class II relative to Class III, IV, and V; Cg09713586_BC21 is hypermethylated in Class II relative to Class III, IV, and V; Cg13350270_BC21 is hypomethylated in Class II relative to Class III, IV, and V′ Cg23059826_BC21 is hypomethylated in Class II relative to Class III, IV, and V; and Cg27416966_BC21 is hypomethylated in Class II relative to Class III, IV, and V

Methods of detecting methylation are also provided. For example, a method of detecting the methylation status at one or more locations in DNA from a human subject can include processing DNA of the sample with a machine-based platform and detecting DNA methylation at and/or close to the genomic sequences associated with one or more CpG loci, e.g., the CpG loci biomarkers provided herein. In some forms, the methods include detecting DNA methylation at and/or close to the genomic sequences associated with any integer number of the CpG loci between 2 and 50. The processing can be one or more of PCR, digital droplet PCR, methylation specific PCR, real time methylation specific PCR, and PCR using a methylated DNA specific binding protein, quantitative PCR, quantitative real-time PCR, microarray, or DNA sequencing. In some forms, the DNA is chemically-modified, e.g., with sodium bisulfite or a ten-eleven translocation (TET) protein. The DNA can be purified prior to processing. The sample can be, for example, a blood sample, a urine sample, or kidney cells or tissue such as a kidney biopsy.

Methods of determining the likelihood of having one or more symptoms of lupus nephritis are also provided. The methods can include detecting methylation at one or more CpG loci in DNA of a sample from the subject and comparing the level of methylation to one or more controls. Symptom can include, but are not limited to, endocapillary hypercellularity, neutrophils/karyorrhexis, fibrinoid necrosis, hyaline deposits, cellular/fibrocellular crescents, interstitial inflammation, total NIH activity index, global glomerulosclerosis, fibrous crescents, tubular atrophy, interstitial fibrosis, total NIH chronicity index, or a combination thereof. The control(s) is typically methylation at one or more of the same CpG loci in a sample from a subject with lupus nephritis whose symptoms have been characterized by kidney biopsy (e.g., histopathology). Typically, a DNA methylation pattern in the subject that is the same or similar to the control(s) indicates the subject has one or more of the same symptoms as the control(s).

Methods of treatment are also provided. The methods typically include treating the subject for the class or group of classes of lupus nephritis that is determined according to the methods provided herein. In some forms, subjects with Class II or less disease are not treated with an immunosuppressant. In some forms, subjects with Class III or higher disease are treated with an immunosuppressant.

In more particular forms, subjects with Class II or less are treated with one or more, optionally all of a renin-angiotensin-aldosterone system (RAAS) blocking agent, hydroxychloroquine, and a calcineurin inhibitor (CNI) or glucocorticoids (GC); subjects with Class III or higher disease are treated with one or more, optionally all of, RAAS blockage, hydroxychloroquine, and additional immunosuppressive (IS) therapy: GC and either cyclophosphamide (CYC) or mycophenolate mofetil (MMF) or CNI or MMF and CNI; subjects with Class IV or higher disease are treated with one or more, optionally all of, RAAS blockade, hydroxychloroquine, GC and either CNI or MMF or CYC or azathioprine (AZA); subjects with Class V or higher disease are treated with chronic kidney disease (CKD) management; or a combination thereof.

### BRIEF DESCRIPTION OF THE DRAWINGS

FIG. 1 is a heat map showing the top 50 loci on the y-axis (right). Lupus class II is represented in the last 3 columns from the left and lupus class III/IV/V is represented in the first 25 columns from the right.

FIGS. 2A-2E are scatterplots demonstrating the beta values (percent methylation) of class II vs. III_IV_V at the top 5 differentially methylated CpG loci.

### DETAILED DESCRIPTION OF THE INVENTION

### I. Definitions

As used herein, the term “methylation” means that a methyl group is attached to a base constituting DNA. In genomic DNA of mammalian cells, in addition to A, C, G, and T, there is the fifth base called 5-methylcytosine (5-mC) with a methyl group attached to the fifth carbon of a cytosine ring. The methylation of 5-methylcytosine occurs only at C of CG dinucleotide (5′-mCG-3′) called CpG. Methylation of CpG inhibits the expression of alu or transposon and a repeating sequence of the genome. In addition, since the 5-mC of the CpG is naturally deaminated to be easily thymine (T), the CpG is a site where most epigenetic changes frequently occur in mammalian cells.

The term “genomic region”, as used herein, generally refers to identified regions of nucleic acid that are identified by their location in the chromosome or another reference such a CpG loci.

The term “CpG islands”, as used herein, generally refers to a contiguous region of genomic DNA that satisfies the criteria of: (1) having a frequency of CpG dinucleotides corresponding to an “Observed/Expected Ratio” greater than about 0.6; and (2) having a “GC Content” greater than about 0.5. CpG islands are typically, but not always, between about 0.2 to about 3 kilobases (kb) in length having a high frequency of CpG sites. CpG islands are found at or near promoters of about 40% of mammalian genes. CpG islands are also found outside of mammalian genes. In some examples, CpG islands are found in exons, introns, promoters, enhancers, inhibitors, and transcriptional regulatory elements. CpG islands may tend to occur upstream of so-called “housekeeping genes”. CpG islands may be said to have a CpG dinucleotide content of at least about 60% of what would be statistically expected. The occurrence of CpG islands at or upstream of the 5′ end of genes may reflect a role in the regulation of transcription, and methylation of CpG sites within the promoters of genes may lead to silencing. Silencing of tumor suppressors by methylation is, in turn, a hallmark of a number of human cancers.

The term “CpG shores”, as used herein, generally refers to regions extending short distances from CpG islands in which methylation may also occur. CpG shores may be found in the region about 0 to 2 kb upstream and downstream of a CpG island.

The term “CpG shelves”, as used herein, generally refers to regions extending short distances from CpG shores in which methylation may also occur. CpG shelves may generally be found in the region between about 2 kb and 4 kb upstream and downstream of a CpG island (e.g., extending a further 2 kb out from a CpG shore).

The term “hemi-methylation” or “hemimethylation”, as used herein, generally refers to the methylation state of a palindromic CpG methylation site, where only a single cytosine in one of the two CpG dinucleotide sequences of the palindromic CpG methylation site is methylated (e.g., 5′-CC.sup.MGG-3′ (top strand): 3′-GGCC-5′ (bottom strand)).

The term “hypermethylation”, as used herein, generally refers to the average methylation state corresponding to an increased presence of 5-mC at one or a plurality of CpG dinucleotides within a DNA sequence of a test DNA sample, relative to the amount of 5-mC found at corresponding CpG dinucleotides within another DNA sample, e.g., a normal control DNA sample or DNA sample from a subject with known disease status.

The term “hypomethylation”, as used herein, generally refers to the average methylation state corresponding to a decreased presence of 5-mC at one or a plurality of CpG dinucleotides within a DNA sequence of a test DNA sample, relative to the amount of 5-mC found at corresponding CpG dinucleotides within another DNA sample, e.g., a normal control DNA sample or DNA sample from a subject with known disease status.

The term “methylation state” or “methylation status”, as used herein, generally refers to the presence or absence of 5-methylcytosine (“5-mC”) at one or a plurality of CpG dinucleotides within a DNA sequence. Methylation states at one or more particular palindromic CpG methylation sites (each having two CpG dinucleotide sequences) within a DNA sequence include “unmethylated,” “fully-methylated” and “hemi-methylated.”

The term “methylated cytosine”, as used herein, generally refers to any methylated forms of the nucleic acid base cytosine that contains a methyl or hydroxymethyl functional group at the 5′ position. Methylated cytosines are known to be regulators of gene transcription in genomic DNA. This term may include 5-methylcytosine and 5-hydroxymethylcytosine.

The term “methylation assay”, as used herein, generally refers to any assay for determining the methylation state of one or more CpG dinucleotide sequences within a sequence of DNA.

The term “MSP” (methylation-specific polymerase chain reaction (PCR)), as used herein, generally refers to a methylation assay, such as that described by Herman et al. Proc. Natl. Acad. Sci. USA 93:9821-9826, 1996, and by U.S. Pat. No. 5,786,146, the contents of each of which are incorporated herein by reference.

The term “methylation converted” or “converted” nucleic acid, as used herein, generally refers to nucleic acid, such as for example DNA, that has undergone a process used to convert the DNA for methylation sequencing. Examples of conversion processes include reagent-based (such as bisulfite) conversion, enzymatic conversion, or combination conversion (such as TET-assisted pyridine borane sequencing (TAPS) conversion), where unmethylated cytosines are converted into uracil prior to PCR amplification or sequencing. The conversion process may be used in methyl sequencing methods to distinguish between methylated and unmethylated cytosine bases.

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. A subject can include a control subject or a test subject.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. The effect of the effective amount can be relative to a control. Such controls are known in the art and discussed herein, and can be, for example the condition of the subject prior to or in the absence of administration of the drug, or drug combination.

The term “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “treating” or “preventing” a disease, disorder, or condition includes ameliorating at least one symptom of the disease or condition. Desirable effects of treatment include decreasing the rate of disease progression, ameliorating, or palliating the disease state, and remission or improved prognosis. For example, an individual is successfully “treated” if one or more symptoms associated with one or more diseases or disorders are mitigated or eliminated, including, but are not limited to, decreasing symptoms resulting from the disease, increasing the quality of life of those suffering from the disease, decreasing the dose of other medications required to treat the disease, delaying the progression of the disease, and/or prolonging survival of individuals.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Biomarkers of Lupus Nephritis Class

Lupus nephritis (LN) is characterized by inflammation of the kidneys and is one of the organ-specific disease manifestations of Systemic Lupus Erythematosus (SLE) (Waldman and Madaio (2005) Lupus 14 (1): 19-24). LN is a chronic inflammatory disease characterized by auto-antibody production and other distinct immunological abnormalities (Gurevitz et al. (2013) Consult Pharm 28:110-21). It is categorized histologically into six classes by the International Society of Nephrology/Renal Pathology Society (ISN/RPS) classification system that has become the standard for renal biopsy interpretation because of improved correlation with prognostic and therapeutic outcomes. (Weening et al. (2004) J Am Soc Nephrol. 15 (2): 241-50; Markowitz et al. (2007) Kidney Int; 71 (6): 491-5). Immune complex formation in LN is the result of systemic autoimmunity and is a hallmark of the disease (Waldman (2005) Lupus 14 (1): 19-24; Nowling (2011) Arthritis Res Ther. 13 (6): 250). Once formed, immune complexes activate complement, which can injure renal cells, leading to either mesangial LN (class I, II), endothelial-proliferative LN (class III, IV), or nephrotic syndrome (class V).

Most patients with SLE who develop LN do so within 5 years of an SLE diagnosis, although it is not uncommon for LN to develop at later times (Anders, et al., Nat Rev Dis Primers 6, 7 (2020). doi.org/10.1038/s41572-019-0141-9, which is specifically incorporated by reference herein in its entirety). In many cases, LN is the presenting manifestation that results in the diagnosis of SLE1. Understanding of the genetic and pathogenetic basis of LN has improved substantially over the past few decades. However, despite this increased knowledge and improved treatment options, LN remains a substantial cause of morbidity and death among patients with SLE. Five-year mortality for LN decreased between 1975 and 1995 but remained stable thereafter, and the rate of progression to end-stage kidney disease (ESKD) remains unchanged. Treatment of LN usually involves immunosuppressive therapy, typically with mycophenolate mofetil or cyclophosphamide and with glucocorticoids, although these treatments are not uniformly effective. Early and accurate diagnosis of LN and prompt initiation of therapy are of vital importance to improve outcomes in patients with SLE.

However, to achieve appropriate diagnosis, treatment, and monitoring, invasive kidney biopsies are routinely performed in patients with lupus nephritis, as the histopathological classification of disease on biopsy guides immunosuppressive treatment. The main risk of kidney biopsies is bleeding, and patients with lupus often have higher risk for bleeding as they can be anemic or thrombocytopenic.

To solve this problem, it has been determined that DNA methylation can be used as an indicator of lupus nephritis class that previously required a biopsy. The experiments provided in the Examples below show that using unbiased hierarchical clustering, methylation in samples clustered based on their histopathological lupus nephritis class (class II samples clustered together, class III samples clustered together, etc.). Furthermore, there was significant differential methylation between the lupus nephritis classes, with significant CpG loci with genomic coordinates identified.

The biomarkers themselves, as well as methods of detecting them not only in kidney samples, but also non-invasive liquid biopsies (e.g., blood and urine), and uses thereof including, but not limited, class diagnosis and subsequent treatment guidance are provided. Due to the less invasive nature of sample collection (e.g., blood and urine), such methods can avoid the risks of conventional kidney biopsies.

### A. Biomarkers of Nephritis Class

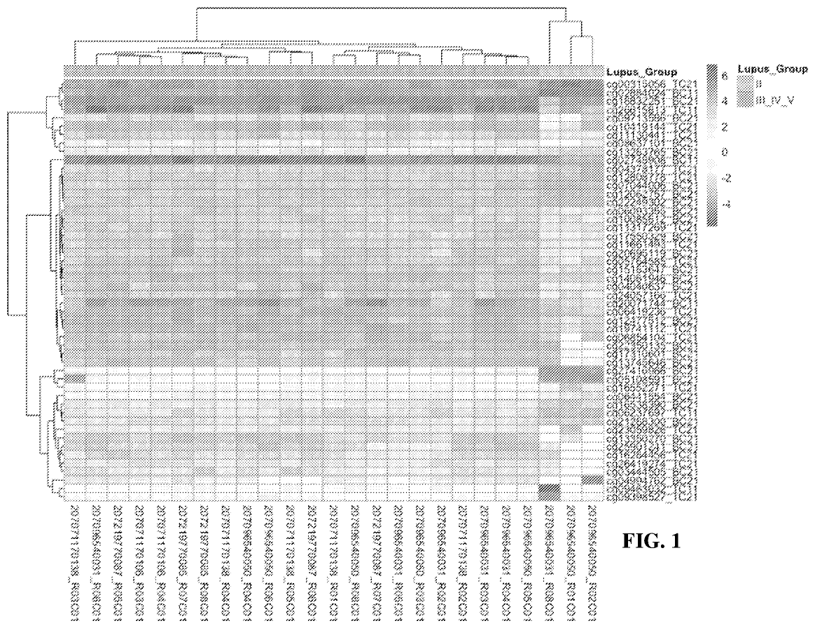

The top 50 differentially methylated CpG loci between patients with lupus nephritis class II and more advanced nephritis (class III, IV, V, III+V, or IV +5) are identified Table 2 and illustrated in the heat map of FIG. 1. Referred to herein as biomarkers, the CpG loci are, in descending order of greatest statistical significance: cg09713586_BC21, cg23059826_TC21, cg13350270_BC21, cg27416966_BC21, cg05108591_BC21, cg04040637_BC21, cg22350135_BC21, cg26419274_TC21, cg12477512_BC21, cg17310601_BC21, cg19741112_TC21, cg11661493_TC21, cg10419144_TC21, cg25901241_BC21, cg08637101_BC21, cg12062757_BC21, cg16552271_TC21, cg00315056_TC21, cg03444505_BC21, cg06093355_BC21, cg04378177_TC21, cg16538390_BC21, cg15183647_BC21, cg04994762_BC21, cg13745646_BC21, cg18832251_BC21, cg20695119_BC21, cg02884024_BC11, cg06441554_BC21, cg04806722_BC21, cg22249302_BC21, cg07044006_BC21, cg05764585_TC21, cg16284456_TC21, cg14061946_BC21, cg09463032_TC11, cg02748908_BC11, cg24057166_TC21, cg13283765_BC21, cg13561626_BC21, cg07642801_BC21, cg11130441_TC21, cg06419236_TC21, cg09398527_TC21, cg10085612_BC21, cg06237697_TC11, cg17550329_BC21, cg26615813_TC11, cg11317269_TC21, and cg21288300_BC21.

As discussed in more detail below, the methods provided herein typically include detection of the methylation level at one or more of the foregoing CpG loci and/or close thereto. Any of the disclosed methods can include analysis of methylation at one or a combination of any 2 or more of the foregoing CpG biomarker sites. Thus, any of the methods can include detection of methylation at any integer number of CpG biomarker sites between 2 and 50 inclusive, in any combination.

The methods typically include detecting DNA methylation at and/or close to the genomic sequences associated with CpG loci. The detection can, but need not, encompass all of CpG at the loci, and can additionally or alternatively include detection of CpG adjacent to, but outside the loci. Such CpG can be referred to as close to the CpG loci. CpG close to the CpG loci are typically not part of another CpG loci. CpG close to the CpG loci may be within, e.g., 10, 100, 1,000, or 10,000 nucleotides, etc., from e.g., the end of the named loci.

In some forms, the methods include detection of methylation at least one, two, three, four, five, six, seven, eight, nine, or all of ten of biomarkers cg09713586_BC21, cg23059826_TC21, cg13350270_BC21, cg27416966_BC21, cg05108591_BC21, cg04040637_BC21, cg22350135_BC21, cg26419274_TC21, cg12477512_BC21, and cg17310601_BC21. In some forms, the methods include detection of methylation at least one, two, three, four, or all five of biomarkers cg09713586_BC21, cg23059826_TC21, cg13350270_BC21, cg27416966_BC21, and cg05108591_BC21. By illustration, as shown in the data presented in the Examples below, Cg05108591_BC21 is hypomethylated in Class II relative to Class III, IV, and V; Cg09713586_BC21 is hypermethylated in Class II relative to Class III, IV, and V; Cg13350270_BC21 is hypomethylated in Class II relative to Class III, IV, and V′ Cg23059826_BC21 is hypomethylated in Class II relative to Class III, IV, and V; and Cg27416966_BC21 is hypomethylated in Class II relative to Class III, IV, and V.

### B. Source of the Samples

The preferred biological sample for detection of the biomarkers is from blood, urine, or kidney-derived tissue including DNA or from which DNA can be isolated. In preferred forms, the DNA is from blood or urine. Thus, in some forms, the biological sample is a liquid biopsy. Such DNA can be extracted from cells or can be cell-free DNA. In some forms, the biological sample is blood or urine, and the DNA is extracted from cells therein, e.g., immune cells and/or other cell debris therein. In some forms, the DNA is extracted from blood buffy-coat. DNA can be purified or isolated from cells or cell-free fluids using any suitable known compositions and methods, including commercially available DNA purification kits.

### C. Methods of Detecting Biomarkers

The methods can include detection of CpG methylation at part or all of the identified CpG loci and/or adjacent genomic regions e.g., 0 to 4 kilobases (kb), about 0 to 3 kb, about 0 to 2 kb, about 0 to 1 kb, about 0 to 500 base pairs (bp), about 0 to 400 bp, about 0 to 300 bp, about 0 to 200 bp, or about 0 to 100 bp upstream and/or downstream thereof. The CpG sequences that are detected can be or include CpG islands, CpG shores, and/or CpG shelves.

The detection can be quantitative or qualitative. In some forms, the detection is absolute, meaning that methylation is detected or not detected. In some forms, the level of methylation is detected. Thus, some of the methods include measuring of the level of methylation level.

The DNA methylation biomarkers can be detected by PCR, digital droplet PCR, methylation specific PCR, real time methylation specific PCR, and PCR using a methylated DNA specific binding protein, quantitative PCR, quantitative real-time PCR, microarray such as DNA chips, beads, or arrays including those sold commercially, sequencing methods such as pyrosequencing, bisulfite sequencing, methylation-specific restriction digestion, mass spectroscopy, methylation-specific immunoassays, and the like, which is known in the art, but is not limited thereto.

Principles and methods of determining CpG site specific methylation and/or differential methylation at the same site(s) in various samples are known in the art. Such methods can include “conversion” of methylated DNA. Exemplary non-limited methods are discussed below, but see also, Kurdyukov and Bullock, Biology (Basel). 2016 March; 5 (1): 3; doi: 10.3390/biology5010003, WO01/26536, US2003/0148326A1, US2023/0132750, US2023/0109129, and US 2023/0101485 each of which is specifically incorporated by reference herein.

The methods can include a bisulfite treatment-dependent detection method or a bisulfite-free detection method. The predominant methodology for DNA methylation analysis relies on the chemical deamination by sodium bisulfite of unmodified cytosine to uracil to permit the differential readout of methylated cytosines. Bisulfite treatment damages the DNA leading to fragmentation and loss of long-range methylation information. To overcome this limitation of bisulfite treated DNA enzymatic deamination approaches have been developed. For example, the methylation level may be measured using a ten-eleven translocation (TET) protein as a bisulfite-free detection method. The TET protein is an enzyme that acts on DNA and is involved in chemical changes of bases.

Since methylated cytosine pairs with guanine in the same way unmethylated cytosine does, traditional sequencing methods (based on base-pairing) are not able to differentiate between methylated and unmethylated cytosines. To solve this problem, sodium bisulfite can be used to convert unmethylated cytosines to uracils, which are amplified as thymines in PCR; because methylated cytosines do not react with sodium bisulfite, they remain as cytosines in the sequence. Thus, thymines detected in bisulfite sequencing or by designing methylation specific (and/or non-methylation specific) primers that correspond to either thymines or unmethylated cytosines in the original DNA, and alignment with the original template sequence (or the presence or absence of PCR product) easily differentiates between them.

More recently, an enzymatic methyl-seq (EM-seq) technique was developed, which uses TET2 in the first enzymatic step to oxidize methylated cytosines and APOBEC2 in the second enzymatic step to convert unmethylated cytosines to uracils. During the subsequent PCR amplification, oxidized methylcytosines form base pairs with guanines and uracils form base pairs with adenines. Since the end products of WGBS and EM-seq (or their PCR-related alternatives) are the same (methylated cytosines stay as cytosines and unmethylated cytosines appear as thymines in the sequence), the same analysis tools can be used. Because enzymatic reactions are non-destructive, EM-seq promises better yield and higher accuracy in the measurement of methylation levels. See, e.g., Feng, et al. Epigenetics & Chromatin 13, 42 (2020). doi.org/10.1186/s13072-020-00361-9, which is specifically incorporated by reference herein in its entirety.

Alternatively, Liu et al. attempted to sequence 5mC and 5hmC directly and to retain unmodified cytosine intact (Liu, Nat Biotechnol 37, 424-429 (2019), doi.org/10.1038/s41587-019-0041-2, which is specifically incorporated by reference herein in its entirety). TET enzymes are used to oxidize 5mC and 5hmC to 5-carboxylcytosine (5caC), and then conversion to dihydrouracil (DHU) is induced via borane reduction. A subsequent PCR reaction facilitates the conversion of DHU to thymine. The researchers termed this 5mC/5hmC-to-T method TET-assisted pyridine borane sequencing (TAPS). An advantage is that the mild reaction conditions can preserve DNA fragments more than 10 kb long. To sequence 5mC alone, they used β-glucosyltransferase to protect 5hmC from TET oxidation and borane reduction so that only 5mC was converted to T. Alternatively, one could use potassium perruthenate to specifically oxidize 5hmC, thus enabling 5hmC-to-T conversion.

When treated with bisulfite all Cs except for methylated C are converted to T bases, whereas with the TET protein, all Cs except for methylated C or only the methylated Cs are converted to T, depending on the downstream steps.

Thus, a preparation for detecting methylation level of the CpG sites may include a compound that modifies a cytosine base or a methylation sensitive restriction enzyme, a primer or primers specific to a methylated sequence, and/or a primer or primers specific to an unmethylated allele sequence.

Pyrosequencing of bisulfite-treated DNA is based on the following principle: when the methylation occurs at a CpG dinucleotide site, 5-methylcytosine (5-mC) is formed, and in this case, the modified base is changed to uracil upon treatment with bisulfite. If the CpG dinucleotide has been methylated when the DNA extracted from the sample is treated with bisulfite, the CpG dinucleotide is preserved as cytosine and the remaining unmethylated cytosines are changed to uracils. Sequencing of the bisulfite-treated DNA may be preferably performed using a pyrosequencing method. The detailed description of pyrosequencing is known in the prior arts (Ronaghi et al, Science 1998 Jul. 17, 281 (5375), 363-365; Ronaghi et al, Analytical Biochemistry 1996 Nov. 1, 242 (1), 84-9; Ronaghi et al. Analytical Biochemistry 2000 Nov. 15, 286 (2): 282-288; Nyr, P. Methods Mol Biology 2007, 373, 114].

Alternatively, by the bisulfite-free detection method using the TET protein, only methylated C may be converted to T using the TET protein to detect the base at the methylated region (see Liu, supra). When the methylation occurs at the CpG dinucleotide site so that cytosine is formed as 5-methylcytosine (5-mC), the CpG dinucleotide has been methylated when treated with the ten-eleven translocation (TET) protein to be changed to uracil, and unmethylated cytosines are preserved.

Detection by bisulfite and/or enzyme-treated DNA is not limited only to the pyrosequencing method, but may be performed by using methods such as PCR, methylation-sensitive PCR (MSP), microarray, next generation sequencing (NGS), and the like.

PCR-based detection methods are also provided for use alone or in combination with other techniques including restriction digestion, sequencing, etc. Such methods can include the use of specifically designed primers. The primers may include a primer(s) specific CpG site(s) being methylated and/or primer(s) specific for site being unmethylated. The term “primer” refers to a nucleic acid sequence having a short free 3-terminal hydroxyl group, and a short nucleic acid sequence capable of forming a base pair with a complementary template and serving as a starting point for copying a template strand. The primer may initiate DNA synthesis in the presence of a reagent for polymerization (i.e., DNA polymerase or reverse transcriptase) and four different nucleoside triphosphates in appropriate buffer and temperature. In addition, the primer is a sense and antisense nucleic acid with a sequence of 7 to 50 nucleotides and may incorporate an additional feature without changing the basic properties of the primer that serves as an initiation site of DNA synthesis.

Primer sequences can be designed for specific CpG sites, and further can be designed for “converted” DNA sequences. In some forms, the detection methods include at least one, and optionally two or more, selected the group consisting of a primer pair capable of specifically amplifying cytosine that is methylated to be unmodified by bisulfite or TET; a primer pair capable of specifically amplifying cytosine that is not methylated to be modified by bisulfite or TET; a primer pair capable of specifically amplifying cytosine that is methylated to be modified by a TET-based protein; and a primer pair capable of specifically amplifying cytosine that is not methylated and has not been modified by a TET-based protein.

Methylation-specific PCR method is a method of designing and using different types of primers depending on whether CpG dinucleotide is methylated in a primer to perform PCR after treating a sample DNA with bisulfite. If a primer binding site has been methylated, PCR is performed by the methylated primer, and if not methylated, PCR is performed by a normal primer. That is, the methylation-specific PCR method is a method of treating the sample DNA with e.g., bisulfite or a TET protein, performing PCR using two types of primers at the same time, and comparing the results.

In real time methylation specific PCR, methylation-specific PCR method is converted to a real-time measurement method and to perform real time PCR by treating genomic DNA with bisulfite or a TET protein, designing PCR primers corresponding to methylation, and using these primers. Examples include, but are not limited to, detection method using a TaqMan probe complementary to an amplified base sequence and a detection method using SYBRgreen. Accordingly, the real time methylation specific PCR may selectively quantitative-analyze only methylated DNA. In this case, the real time methylation specific PCR is a method of preparing a standard curve using an in vitro methylated DNA sample, and quantitatively analyzing the methylation level by amplifying a target CpG site biomarker together as a negative control for standardization.

In an exemplary assay, chemically-modified DNA is seeded into quantitative real time PCR reactions that contain sequence-specific PCR primers for DNA amplification and sequence-specific probes to detect the target sequences, in addition to standard amplification reagents. The sequence-specific probes may contain locked nucleic acids to increase specificity and sensitivity of detection, and can be fluorescently labeled to allow for quantitation of the PCR products being amplified. This assay can be performed in any suitable format including, but not limited to, a multiplexed 96-well format. Following PCR, a post-hoc analysis of the assay can performed and a report can be prepared for the physician that indicates the results of the test. Digital droplet PCR is another technology platform that holds a certain level of appeal for analysis of DNA over that of quantitative real time PCR.

In addition or alternative, the methods can include use of a methylation-sensitive restriction enzyme. The methylation-sensitive restriction enzyme may be a restriction enzyme capable of specifically detecting the methylation of the CpG site, and may be a restriction enzyme containing CG as a recognition site of the restriction enzyme. For example, the methylation-sensitive restriction enzyme includes Smal, SacII, EagI, HpaII, MspI, BssHII, BstUI, NotI, and the like, but is not limited thereto. Depending on methylation or unmethylation at C of the restriction enzyme recognition site, cleavage by the restriction enzyme varies and may be detected through PCR or southern blot analysis. In a method for measuring the methylation using the methylation-sensitive restriction enzyme, the methylation-sensitive restriction enzyme uses CpG dinucleotide as an action site, and does not act as the enzyme when this site is methylated. Therefore, when the sample DNA is treated with the methylation-sensitive restriction enzyme and then amplified by PCR so as to include an enzyme target site, in the case of the methylated site, the restriction enzyme does not act, but is amplified by PCR, but the unmethylated normal site is cleaved by the restriction enzyme and not amplified by PCR, thereby measuring the methylation of a specific DNA site.

Nucleic acid chips for use in detection of the provided CpG biomarkers can include immobilized probes capable of hybridizing with fragments including CpG sites of one or more or biomarkers. In some forms, such probes are design to bind “converted” DNA.

Some methods include using a methylated DNA-specific binding protein. When a protein that specifically binds only to methylated DNA is mixed with DNA, the protein specifically binds only to the methylated DNA, so that only the methylated DNA may be selectively isolated. After mixing genomic DNA with the methylated DNA-specific binding protein, only the methylated DNA is selectively isolated. In some forms, the methods include amplifying these isolated DNAs using a PCR primer corresponding to a biomarker site, and then measuring the methylation by agarose electrophoresis. In addition, the methylation may be measured even by quantitative PCR, and the methylated DNA isolated by the methylated DNA-specific binding protein is labeled with a fluorescent dye or hybridized to a DNA chip integrated with a complementary probe to measure the methylation.

### D. Diagnosis

### 1. Single Markers

The biomarkers can be used in diagnostic and prognostic tests to assess lupus nephritis class in a subject, e.g., a specific class or between less (i.e., class II) and more advanced lupus nephritis (class III, IV, V, III+V, or IV +5), the progress of disease (e.g., progress of disease or remission of disease over time), and the effectiveness or response to treatment of disease. Based on this status, further procedures may be indicated, including additional diagnostic tests or therapeutic procedures or regimens. Representative therapies are discussed in more detail below.

The method involves, first, measuring one or more biomarkers (i.e., methylation at one or more of the disclosed CpG loci and/or the genomic sequence adjacent thereto) in a subject sample using, e.g., the methods described herein, and, second, comparing the measurement with a diagnostic amount or cut-off that distinguishes a positive or negative lupus nephritis status, or specifically identifies the lupus nephritis class. The diagnostic amount represents a measured amount of a biomarker above or below which a subject is classified as having a particular status. For example, if the biomarker is increased (i.e., hypermethylation) in more advanced cases compared to less advanced cases, then a measured amount above the diagnostic cutoff provides a diagnosis of a more advanced case. Likewise, if the biomarker is decreased (hypomethylation) in more advanced cases compared to less advanced cases, then a measured amount below the diagnostic cutoff provides a diagnosis of a more advanced case. Similarly, if the biomarker is increased (i.e., hypermethylation) in less advanced cases compared to more advanced cases, then a measured amount above the diagnostic cutoff provides a diagnosis of a less advanced case. Likewise, if the biomarker is decreased (hypomethylation) in less advanced cases compared to more advanced cases, then a measured amount below the diagnostic cutoff provides a diagnosis of a less advanced case.

In other forms, the test measurement is compared directly to measurements from subjects each with a known lupus nephritis status, representing two or more different lupus nephritis classes or categories. Each class of the two or more different classes or category (of less or more advanced lupus nephritis) can be a single measurement or an average of pooled measures from subjects of the same class or category. The test subject's status can be determined selecting that class (or range of classes) of the known subject(s) that most closely matches test measurement.

As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different lupus nephritis statuses and drawing the cut-off to suit the diagnostician's desired levels of specificity and sensitivity.

### 2. Combinations of Markers

While individual biomarkers are useful diagnostic biomarkers, a combination of biomarkers may provide greater predictive value of a particular status than single biomarkers alone. Specifically, the detection of a plurality of biomarkers in a sample can increase the sensitivity and/or specificity of the test. Thus, in one form, two or more, three or more, four or more or even five or more of the biomarkers can be detected and used to assess the status of lupus nephritis in a subject. In some forms, the biomarkers include methylation at one or more of cg09713586_BC21, cg23059826_TC21, cg13350270_BC21, cg27416966_BC21, cg05108591_BC21, cg04040637_BC21, cg22350135_BC21, cg26419274_TC21, and/or cg12477512_BC21, cg17310601_BC21, or more preferably one or more of cg09713586_BC21, cg23059826_TC21, cg13350270_BC21, cg27416966_BC21, and/or cg05108591_BC21.

### E. Determining Risk of Developing More Severe Disease

Methods for determining the risk of developing more severe disease in a subject are also provided. Biomarker amounts or patterns can be characteristic of various risk states, e.g., high, medium, or low. The risk of developing a disease is determined by measuring the biomarker or biomarkers and then either submitting them to a classification algorithm or comparing them with a reference amount and/or pattern of biomarkers that is associated with the particular risk level.

### F. Determining Stage of Disease

As introduced above, some forms provide methods for determining the specific stage of disease in a subject. Each stage of the disease can have a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage. The stage can be a category such as less advanced (e.g., class II or lower) or more advanced disease (e.g., higher than class II), or a specific class.

Results in Example 2 also show that DNA methylation patterns from immune cells correlate with certain histopathologic findings, particularly crescent formation, from kidney biopsy in patients with lupus nephritis. Thus, in some forms, the methods inform likely pathologies without the need for a biopsy.

### G. Determining Course (Progression/Remission) of Disease

Still another form provides methods for determining the course of disease in a subject. Disease course refers to changes in disease status over time, including disease progression (worsening) and disease regression (improvement). Over time, the amounts or relative amounts (e.g., the pattern) of the biomarkers changes. This method involves measuring one or more biomarkers in a subject at at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal, while if treatment is ineffective, the biomarkers will trend toward disease indications.

### H. Subject Management

In certain forms of the method including the detection and/or analysis of one or more biomarkers further include managing subject treatment based on the status. Such management includes the actions of the physician or clinician subsequent to determining lupus nephritis status. For example, if a physician makes a determination of lupus nephritis status, then a certain regime of treatment might follow. Alternatively, a determinate or an indeterminate result might be followed with further testing to determine further testing, e.g., a kidney biopsy.

One form provides a method for selecting a subject for treatment for lupus nephritis by detecting the presence or quantity of one or more biomarkers provided herein in a sample from a subject having or suspected of having lupus nephritis, comparing the levels of biomarker in the sample to a predetermined standard, wherein the subject is selected for treatment based on a determination of the lupus nephritis status. As discussed in more detail below, Disease classification, based on the disclosed methods, provides clinically actionable information, as class II disease typically does not require kidney-specific immunosuppression, while classes III, IV, or V disease typically do.

Additional forms relate to the communication of assay results or diagnoses or both to technicians, physicians or patients, for example. In certain forms, computers will be used to communicate assay results or diagnoses or both to interested parties, e.g.: physicians and their patients. In some forms, the assays will be performed or the assay results analyzed in a country or jurisdiction which differs from the country or jurisdiction to which the results or diagnoses are communicated.

In a preferred form a diagnosis based on the presence or absence in a test subject of any the disclosed biomarkers is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain forms, the message containing results of a diagnostic test may be generated and delivered automatically to the subject using a combination of computer hardware and software which will be familiar to artisans skilled in telecommunications. In certain forms all or some of the method steps, including the assaying of samples, diagnosing of diseases, and communicating of assay results or diagnoses, may be carried out in diverse (e.g., foreign) jurisdictions.

### I. Assessing the Effectiveness of Treatment

Methods for determining the course of lupus nephritis in a subject are also provided. Disease course refers to changes in disease status over time, including disease progression (worsening) and disease regression (improvement). Over time, the amounts or relative amounts (e.g., the pattern) of the biomarkers changes. Accordingly, this method involves measuring one or more biomarkers in a subject at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal or not further worsen, while if treatment is ineffective, the biomarkers will trend toward more severe disease indications.

### III. Methods of Treatment

Method of treatment lupus nephritis are also provided. Such methods can be used in conjunction with any one or more of the disclosed methods otherwise provided herein, e.g., the methods of detecting one or more biomarkers indicative of lupus nephritis class, and their associated uses. Prompt diagnosis and early institution of specific treatment is essential to improve outcomes in lupus nephritis (LN). All patients with systemic lupus erythematosus (SLE) should be regularly screened for LN.

Common treatments include, but are not limited to:

Diet changes, which can limit the amount of protein and salt to improve kidney function.

Blood pressure medications such as angiotensin-converting enzyme (ACE) inhibitors and angiotensin II receptor blockers (ARBs), which can help control blood pressure and prevent protein from leaking from the kidneys into the urine.

Diuretics, which can help eliminate excess fluid.

Immunosuppressant and anti-inflammatory drugs, which can reduce inflammatory and/or autoimmunity, such as steroids, such as prednisone, cyclosporine, tacrolimus, cyclophosphamide, Azathioprine (Imuran), Mycophenolate (CellCept), Rituximab (Rituxan), Belimumab (Benlysta).

Treatments for kidney failure such as dialysis and/or kidney transplant.

See, also Table 2 of Anders, et al., Nat Rev Dis Primers 6, 7 (2020). doi.org/10.1038/s41572-019-0141-9, which is specifically incorporated by reference herein in its entirety.

Treatment is initiated and maintained or adjusted based on disease class. See, e.g., Anders, supra. For example, class II disease does not require kidney-specific immunosuppression, while classes III, IV, or V disease do.

For example, in some forms, subjects with a Class II or less diagnosis are treated with one or more, optionally all of an renin-angiotensin-aldosterone system (RAAS) blocking agent, hydroxychloroquine, and a calcineurin inhibitor (CNI) or glucocorticoids (GC).

In some forms, subjects with a Class III, Class IV, or higher diagnosis are treated with one or more, optionally all of, RAAS blockage, hydroxychloroquine, and additional immunosuppressive (IS) therapy: GC and either cyclophosphamide (CYC) or mycophenolate mofetil (MMF) or CNI or MMF and CNI.

In some forms, subjects with a Class IV, or higher diagnosis are treated with one or more, optionally all of, RAAS blockade, hydroxychloroquine, GC and either CNI or MMF or CYC or azathioprine (AZA).

In some forms, subjects with a Class VI, or higher diagnosis are treated with chronic kidney disease (CKD) management.

The role of immunosuppressive (IS) therapy in treating class I and class II LN is not clear and most guidelines do not recommend its routine use in these subject. Thus, in some forms, subjects determined to have class II or less are not treated with an IS therapy. The KDIGO guidelines propose treatment with glucocorticoids (GCs) or CNIs only in the subset of patients with podocytopathy and nephrotic syndrome. However, the European League Against Rheumatism guidelines recommend low-to-moderate dosages of oral glucocorticoids (0.25-0.5 mg/kg daily), alone or in combination with azathioprine (1-2 mg/kg daily), in cases of proteinuria with protein excretion exceeding 1 g daily. Thus, in some forms, the subject diagnosed according to the disclosed methods are further tested for proteinuria and protein excretion and optionally the treatments are adjusted accordingly.

All patients should also receive adjunctive treatment and supportive care.

The disclosed invention can be further understood by the following numbered paragraphs:
1. A method of detecting the methylation status at one or more locations in DNA from a human subject including processing DNA of a sample from the subject with a machine-based platform and detecting DNA methylation at and/or close to the genomic sequences associated with one or more CpG loci selected from the group consisting of cg09713586_BC21, cg23059826_TC21, cg13350270_BC21, cg27416966_BC21, cg05108591_BC21, cg04040637_BC21, cg22350135_BC21, cg26419274_TC21, cg12477512_BC21, cg17310601_BC21, cg19741112_TC21, cg11661493_TC21, cg10419144_TC21, cg25901241_BC21, cg08637101_BC21, cg12062757_BC21, cg16552271_TC21, cg00315056_TC21, cg03444505_BC21, cg06093355_BC21, cg04378177_TC21, cg16538390_BC21, cg15183647_BC21, cg04994762_BC21, cg13745646_BC21, cg18832251_BC21, cg20695119_BC21, cg02884024_BC11, cg06441554_BC21, cg04806722_BC21, cg22249302_BC21, cg07044006_BC21, cg05764585_TC21, cg16284456_TC21, cg14061946_BC21, cg09463032_TC11, cg02748908_BC11, cg24057166_TC21, cg13283765_BC21, cg13561626_BC21, cg07642801_BC21, cg11130441_TC21, cg06419236_TC21, cg09398527_TC21, cg10085612_BC21, cg06237697_TC11, cg17550329_BC21, cg26615813_TC11, cg11317269_TC21, and cg21288300_BC21.
2. The method of paragraph 1 including detecting DNA methylation at and/or close to the genomic sequences associated with one or more CpG loci selected from the group consisting of cg09713586_BC21, cg23059826_TC21, cg13350270_BC21, cg27416966_BC21, cg05108591_BC21, cg04040637_BC21, cg22350135_BC21, cg26419274_TC21, cg12477512_BC21, and cg17310601_BC21.
3. The method of paragraphs 1 or 2 including detecting DNA methylation at and/or close to the genomic sequences associated with one or more CpG loci selected from the group consisting of cg09713586_BC21, cg23059826_TC21, cg13350270_BC21, cg27416966_BC21, and cg05108591_BC21.
4. The method of any one of paragraphs 1-3 including detecting DNA methylation at and/or close to the genomic sequences associated with any integer number of the CpG loci between 2 and 50.
5. The method of any one of paragraphs 1-4, wherein the processing includes one or more of PCR, digital droplet PCR, methylation specific PCR, real time methylation specific PCR, and PCR using a methylated DNA specific binding protein, quantitative PCR, quantitative real-time PCR, microarray, or DNA sequencing.
6. The method of any one of paragraphs 1-5, wherein the DNA is chemically-modified.
7. The method of paragraph 6, wherein the DNA is chemically modified with sodium bisulfite or a ten-eleven translocation (TET) protein.
8. The method of any one of paragraphs 1-6, wherein the DNA is purified prior to processing with the machine-based platform.
9. The method of paragraph 1, wherein sample is a blood sample, a urine sample, or kidney cells or tissue optionally a kidney biopsy.
10. A method of determining lupus nephritis class including detecting methylation at one or more differentially methylated CpG loci and comparing the level of methylation to one or more controls.
11. The method of paragraph 10, wherein the control(s) are known methylation values for the same CpG loci in subjects previously categorized as being Class II or less, or Class III or higher.
12. The method of paragraph 10, wherein the control(s) are known methylation values for the same CpG loci in subjects previously diagnosed as being Class II, Class III, Class IV, or Class V.
13. The method of any one of paragraphs 10-12, wherein methylation is detected according the method of any one of paragraphs 1-9.
14. The method of any one of paragraphs 10-13, including detecting methylation at one or more of cg09713586_BC21, cg23059826_TC21, cg13350270_BC21, cg27416966_BC21, and cg05108591_BC21, wherein Cg05108591_BC21 is hypomethylated in Class II relative to Class III, IV, and V; Cg09713586_BC21 is hypermethylated in Class II relative to Class III, IV, and V; Cg13350270_BC21 is hypomethylated in Class II relative to Class III, IV, and V′ Cg23059826_BC21 is hypomethylated in Class II relative to Class III, IV, and V; and Cg27416966_BC21 is hypomethylated in Class II relative to Class III, IV, and V.
15. The method of any one of paragraphs 10-14, including treating the subject for the class of lupus nephritis that is determined.
16. The method of paragraph 15, wherein subjects with Class II or less disease are not treated with an immunosuppressant.
17. The method of paragraphs 15 or 16, wherein subjects with Class III or higher disease are treated with an immunosuppressant.
18. The method of any one of paragraphs 15-17, wherein subjects with Class II or less are treated with one or more, optionally all of a renin-angiotensin-aldosterone system (RAAS) blocking agent, hydroxychloroquine, and a calcineurin inhibitor (CNI) or glucocorticoids (GC).
19. The method of any one of paragraphs 15-18, wherein subjects with Class III or higher disease are treated with one or more, optionally all of, RAAS blockage, hydroxychloroquine, and additional immunosuppressive (IS) therapy: GC and either cyclophosphamide (CYC) or mycophenolate mofetil (MMF) or CNI or MMF and CNI.
20. The method of any one of paragraphs 15-19, wherein subjects with Class IV or higher disease are treated with one or more, optionally all of, RAAS blockade, hydroxychloroquine, GC and either CNI or MMF or CYC or azathioprine (AZA).
21. The method of any one of paragraphs 15-20, wherein subjects with Class V or higher disease are treated with chronic kidney disease (CKD) management.
22. A method of determining the likelihood of having one or more symptoms of lupus nephritis in a subject including detecting methylation at one or more CpG loci in DNA of a sample from the subject and comparing the level of methylation to one or more controls.
23. The method of paragraph 22, wherein the symptom is endocapillary hypercellularity, neutrophils/karyorrhexis, fibrinoid necrosis, hyaline deposits, cellular/fibrocellular crescents, interstitial inflammation, total NIH activity index, global glomerulosclerosis, fibrous crescents, tubular atrophy, interstitial fibrosis, total NIH chronicity index, or a combination thereof.
24. The method of paragraph 23, wherein the control(s) is methylation at one or more of the same CpG loci in a sample from a subject with lupus nephritis whose symptoms have been characterized by kidney biopsy.
25. The method of paragraph 24, wherein a DNA methylation pattern in the subject that is the same or similar to the control(s) indicates the subject has one or more of the same symptoms as the control(s).
26. The method of any one of paragraphs 22-25, wherein the DNA sample is from blood, optionally the buffy coat, optionally immune cells therein.
27. The method of any one of paragraphs 22-26, wherein the detecting is according to the method of any one of paragraphs 1-9.
28. The method of any one of paragraphs 22-26, further including treating the subject for one or more of the symptoms.
29. A method of treating a subject for lupus nephritis including administering the subject a treatment informed by the subject's disease class as determined according to the method any one of paragraphs 10-14 or symptoms determined according to the method of any one of paragraphs 22-27.
30. A composition or method as described by any of the text and/or figures provided herein.

### EXAMPLES

### Example 1: Differential Methylation Indicates Lupus Class

### Materials and Methods

### Methylation Analysis

DNA was extracted from blood buffy-coat of 25 participants with biopsy-confirmed lupus nephritis from the Yale Kidney Biobank. Bisulfite conversion was then performed on 200 ng of DNA. Infinium Methylation EPIC array V2.0 was used to survey approximately 935,000 CpG loci. IDAT files were obtained, and β-values were derived using Sesame R package. β-values were logit-transformed to M-values. Differentially methylated loci in participants with class II (n=3) vs. more advanced nephritis (class III/IV/V n=22) were evaluated. Benjamini-Hochberg method was implemented for false discovery rate correction. KEGG pathway enrichment analysis was also performed to identify the top differentially methylated pathways.

TABLE 1
Baseline characteristics of participants from the Yale
Kidney Biobank with lupus nephritis.

| Lupus | | | | | | |
|---|---|---|---|---|---|---|
| Nephritis | II | III | IV | V | III + V | IV + V |
| Class | (n = 3) | (n = 5) | (n = 4) | (n = 9) | (n = 3) | (n = 1) |
| Age (years) | 42 [30.5, 46] | 42 [36, 53] | 25.5 [24.8, 26.5] | 41 [31, 53] | 31 [25, 30] | 35 |
| Male Sex | 0 (0) | 0 (0) | 0 (0) | 1 (11) | 1 (33) | 1 (100) |
| BMI (kg/m²) | 23.9 [23, 27.3] | 37.1 [25.1, 38.2] | 27.4 [25.2, 30.7] | 33 [26.7, 39.3] | 20.7 [19.4, 23.6] | 29.8 |
| uPCR | 1.9 [1.5, 4.7] | 1.2 [0.6, 1.8] | 4.7 [2.9, 5.7] | 2.5 [2.4, 5.4] | 1.7 [1.4, 2] | 14.1 |
| (mg/mg) | | | | | | |
| ACR (mg/g) | 878.1 (n = 1) | 472.2 [244.3, 700] | 924.5 [855.8, 993.3] | 1802.6 [1565.3, 3970.8] | 928.7 [831.1, 1176] | 7080 |
| | | (n = 2) | (n = 2) | (n = 5) | | |
| eGFR | 94 [79, 113.5] | 98 [45, 111] | 58.5 [19.8, 96.8] | 51 [17, 102] | 96 [54, 110] | 14 |
| (ml/min/1.73²) | | | | | | |
| Platelets | 306 [282, 391] | 214 [192, 257] | 207.5 [177.3, 224.8] | 229 [206, 293] | 313 [302, 321.5] | 523 |
| (×1000/μL) | | | | | | |
| Hemoglobin | 10.3 [9.7, 10.3] | 9.9 [9.3, 10.9] | 10.3 [9.1, 11.1] | 8.9 [7.7, 12.2] | 10.6 [9.3, 11.1] | 9.6 |
| (g/μL) | | | | | | |
| WBC Count | 5.1 [4.7, 6.9] | 6.7 [6.4, 7.5] | 4.2 [3.7, 6.4] | 5.8 [4.7, 9.8] | 8.9 [8.15, 9] | 26.8 |
| (thousand | | | | | | |
| d/μL) | | | | | | |
| C3 (mg/dL) | 93 [76.5, 95.5] | 72 [57, 103] | 65.5 [50.5, 80.5] | 100 [95, 110] | 67 [51.5, 69.5] | 67 |
| C4 (mg/dL) | 16 [13, 19] | 13 [8, 23] | 11.5 [7.3, 17] | 15 [15, 31] | 6 [4.5, 9.5] | 13 |

Abbreviations: BMI, body mass index; uPCR, urine protein/creatinine ratio; ACR, albumin to creatinine ratio; WBC, white blood cell.

### LogFC Interpretation

logFC values are calculated as the differences in methylation levels between the “III_IV_V” group and the “II” group. Therefore, a positive logFC indicates higher methylation in “III_IV_V” compared to “II”, and a negative logFC indicates lower methylation in “III_IV_V” compared to “II”. Fold change is a measure that describes how much a quantity changes between an original and a subsequent measurement. In this context, it is used to describe the change in methylation levels between two groups. When fold changes are represented on a logarithmic scale (as is the case with logFC), a value of 0 would mean no change, a value of 1 would correspond to a 2-fold increase (since 2\hat{ }1=2), a value of −1 would correspond to a 2-fold decrease (since 2\hat{ }−1=0.5), and so on.

### Results

The top 50 differentially methylated CpG loci between patients with lupus nephritis class II and more advanced nephritis (class III, IV, V, III+V, or IV +5) were identified, and are presented in Table 2. The top five CpG loci that were differentially methylated between participants with class II nephritis and those with III, IV, or V has adjusted p-values of <0.05.

TABLE 2
Top 50 differentially methylated CpG loci between patients with lupus nephritis class II
and more advanced nephritis (class III, IV, V, III + V, or IV + 5) in descending order.

| CpG loci | logFC | AveExpr | t | P.Value | adj.P.Val | B |
|---|---|---|---|---|---|---|
| cg09713586_BC21 | −4.808335093 | −2.054575751 | −13.38289324 | 5.00E−11 | 4.32E−05 | −2.445152143 |
| cg23059826_TC21 | 3.669784229 | 2.056323962 | 10.25412878 | 9.32E−11 | 4.32E−05 | −1.789697928 |
| cg13350270_BC21 | 2.2759616 | 3.28142194 | 7.637824454 | 3.49E−08 | 0.010790073 | −2.240658312 |
| cg27416966_BC21 | 4.908857138 | −0.288391476 | 7.29878783 | 8.02E−08 | 0.018617473 | −2.319565541 |
| cg05108591_BC21 | 3.912066158 | −1.10459352 | 6.879158608 | 2.29E−07 | 0.042596281 | −2.425352969 |
| cg04040637_BC21 | 1.318229553 | 3.769745954 | 6.354981535 | 8.77E−07 | 0.135687676 | −2.571061737 |
| cg22350135_BC21 | 2.527485824 | 3.918215179 | 6.005983284 | 2.18E−06 | 0.288434384 | −2.676947244 |
| cg26419274_TC21 | 1.729621741 | 2.467277048 | 5.900072865 | 2.87E−06 | 0.333187708 | −2.710533063 |
| cg12477512_BC21 | 1.072591513 | 4.146011656 | 5.752302231 | 4.24E−06 | 0.437012789 | −2.758541052 |
| cg17310601_BC21 | 1.833356608 | 3.501014084 | 5.590398147 | 6.50E−06 | 0.55240528 | −2.81268618 |
| cg19741112_TC21 | 0.970311264 | 4.196853957 | 5.587775415 | 6.55E−06 | 0.55240528 | −2.813576639 |
| cg11661493_TC21 | 1.049446679 | 3.55304276 | 5.353135527 | 1.22E−05 | 0.944310062 | −2.894964268 |
| cg10419144_TC21 | −1.451080196 | −3.540664353 | −5.245290776 | 1.63E−05 | 0.999635267 | −2.933512838 |
| cg25901241_BC21 | 2.38674961 | 2.962048536 | 5.222268198 | 1.73E−05 | 0.999635267 | −2.941834824 |
| cg08637101_BC21 | −1.246244735 | −1.822969423 | −5.183075161 | 1.92E−05 | 0.999635267 | −2.9560767 |
| cg12062757_BC21 | −0.571990578 | 3.769706382 | −5.167180895 | 2.00E−05 | 0.999635267 | −2.961879111 |
| cg16552271_TC21 | 1.471798825 | −0.871888186 | 5.137488674 | 2.17E−05 | 0.999635267 | −2.972759964 |
| cg00315056_TC21 | 0.638133672 | −4.545173758 | 5.12131653 | 2.27E−05 | 0.999635267 | −2.978708922 |
| cg03444505_BC21 | 2.060208394 | 2.530100147 | 5.104005047 | 2.37E−05 | 0.999635267 | −2.985094616 |
| cg06093355_BC21 | 0.871377632 | 3.468571873 | 5.10091705 | 2.39E−05 | 0.999635267 | −2.9862356 |
| cg04378177_TC21 | −0.664614878 | 3.460660521 | −5.094464009 | 2.43E−05 | 0.999635267 | −2.988621803 |
| cg16538390_BC21 | −0.866700089 | 2.706316522 | −5.082079147 | 2.52E−05 | 0.999635267 | −2.993208546 |
| cg15183647_BC21 | 0.818259195 | 3.770645379 | 5.062439202 | 2.65E−05 | 0.999635267 | −3.000501262 |
| cg04994762_BC21 | 3.790806659 | 1.549733987 | 4.98227339 | 3.28E−05 | 0.999635267 | −3.030509709 |
| cg13745646_BC21 | 1.786855212 | 3.737298817 | 4.959181405 | 3.49E−05 | 0.999635267 | −3.039225244 |
| cg18832251_BC21 | 0.763382056 | 4.744420408 | 4.947995139 | 3.60E−05 | 0.999635267 | −3.043458682 |
| cg20695119_BC21 | 1.164174034 | 3.515865776 | 4.906749855 | 4.02E−05 | 0.999635267 | −3.059132218 |
| cg02884024_BC11 | 0.719573662 | −4.345512253 | 4.902228336 | 4.07E−05 | 0.999635267 | −3.060856564 |
| cg06441554_BC21 | −1.141083896 | 1.80739994 | −4.876344988 | 4.36E−05 | 0.999635267 | −3.070750776 |
| cg04806722_BC21 | −3.238330806 | −0.441867845 | −9.291814083 | 4.51E−05 | 0.999635267 | −4.184953092 |
| cg22249302_BC21 | −0.536568034 | 3.773970438 | −4.83272665 | 4.90E−05 | 0.999635267 | −3.087513485 |
| cg07044006_BC21 | −0.709668093 | 3.699045206 | −4.831432616 | 4.92E−05 | 0.999635267 | −3.088012488 |
| cg05764585_TC21 | 0.847499532 | 3.742350595 | 4.826790934 | 4.98E−05 | 0.999635267 | −3.08980321 |
| cg16284456_TC21 | 1.894913334 | 2.863882037 | 4.826612827 | 4.98E−05 | 0.999635267 | −3.089871947 |
| cg14061946_BC21 | 0.660605283 | 3.950932898 | 4.819514649 | 5.08E−05 | 0.999635267 | −3.092612866 |
| cg09463032_TC11 | 2.934774081 | 0.421105717 | 4.805160578 | 5.27E−05 | 0.999635267 | −3.098164563 |
| cg02748908_BC11 | 1.240870181 | 5.917041321 | 4.786444787 | 5.55E−05 | 0.999635267 | −3.105421191 |
| cg24057166_TC21 | 1.115813087 | 3.622381513 | 4.76459692 | 5.88E−05 | 0.999635267 | −3.113917824 |
| cg13283765_BC21 | 0.744145752 | −2.07250677 | 4.69432549 | 7.09E−05 | 0.999635267 | −3.141431924 |
| cg13561626_BC21 | 3.011821912 | 2.508623801 | 10.13714879 | 7.30E−05 | 0.999635267 | −4.365726329 |
| cg07642801_BC21 | −3.325916879 | −1.043539262 | −10.12134462 | 7.36E−05 | 0.999635267 | −4.365772988 |
| cg11130441_TC21 | −1.026980113 | −2.987182704 | −4.672142352 | 7.53E−05 | 0.999635267 | −3.150175772 |
| cg06419236_TC21 | 0.760630327 | 4.340527121 | 4.671246589 | 7.55E−05 | 0.999635267 | −3.150529434 |
| cg09398527_TC21 | 2.625452182 | 0.909733064 | 4.654304164 | 7.89E−05 | 0.999635267 | −3.157227075 |
| cg10085612_BC21 | 0.701107766 | 3.409939266 | 4.653855801 | 7.90E−05 | 0.999635267 | −3.157404539 |
| cg06237697_TC11 | −1.16107872 | 2.927410654 | −4.653780533 | 7.91E−05 | 0.999635267 | −3.157434332 |
| cg17550329_BC21 | 1.047213091 | 3.412475284 | 4.639672842 | 8.21E−05 | 0.999635267 | −3.163024031 |
| cg26615813_TC11 | −1.272965096 | −4.68475384 | −4.636887185 | 8.27E−05 | 0.999635267 | −3.164129067 |
| cg11317269_TC21 | 0.645300121 | 3.384711311 | 4.635312782 | 8.31E−05 | 0.999635267 | −3.164753805 |
| cg21288300_BC21 | −0.62594617 | 3.101336746 | −4.632586768 | 8.37E−05 | 0.999635267 | −3.165835839 |

The heat map in FIG. 1 demonstrates the top 50 loci on the y-axis (right). Lupus class II is represented in aqua/blue (last 3 columns from the left) and lupus class III/IV/V is represented in pink (first 25 columns from the left). The heat map scale illustrates a range from hypomethylation (−4) to hypermethylation (6) of Class II as compared to Class III, IV, and V. For example, a −4 is hypomethylation in class II as compared to III_IV_V.

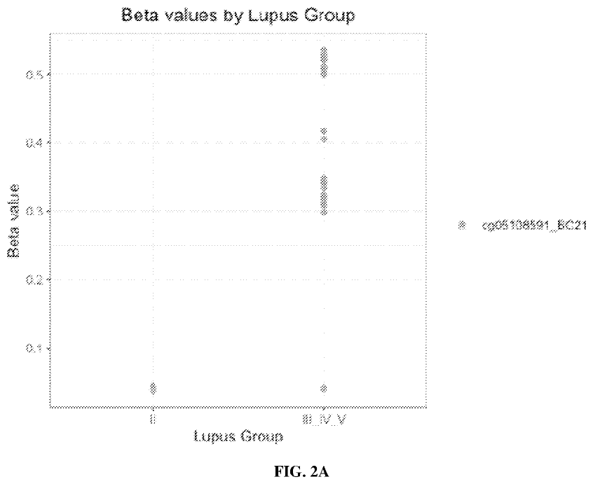

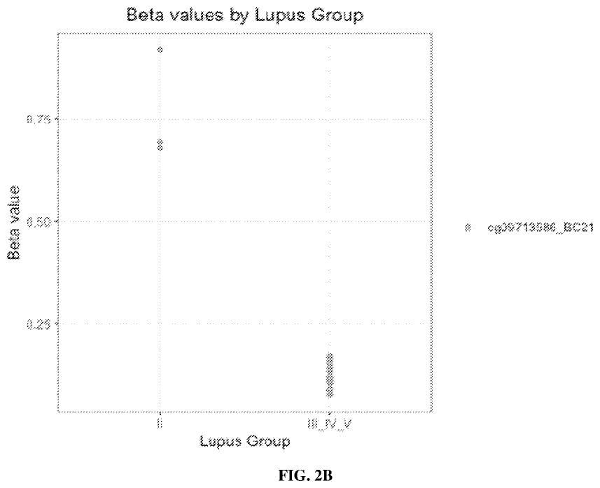

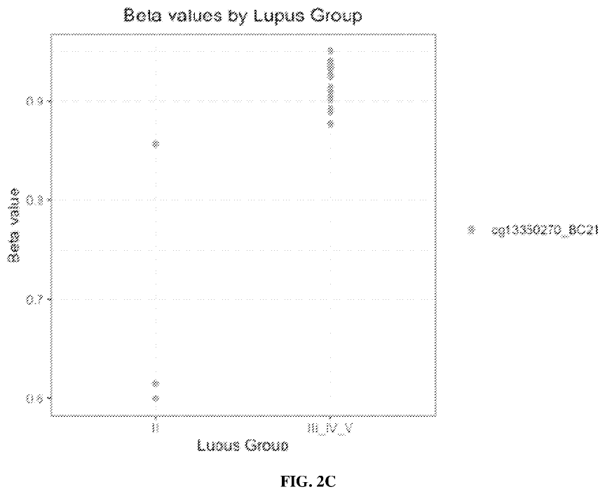

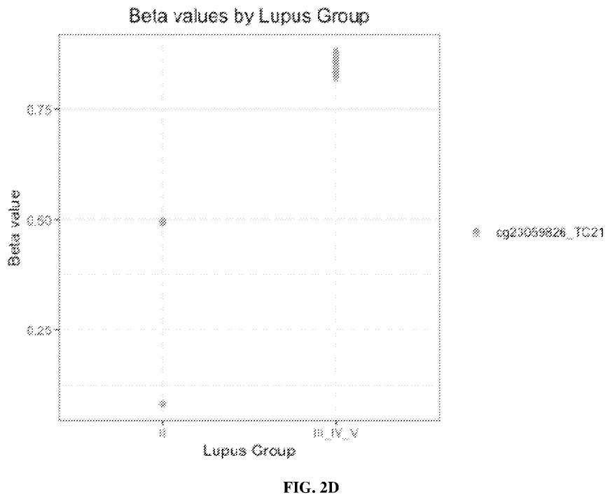

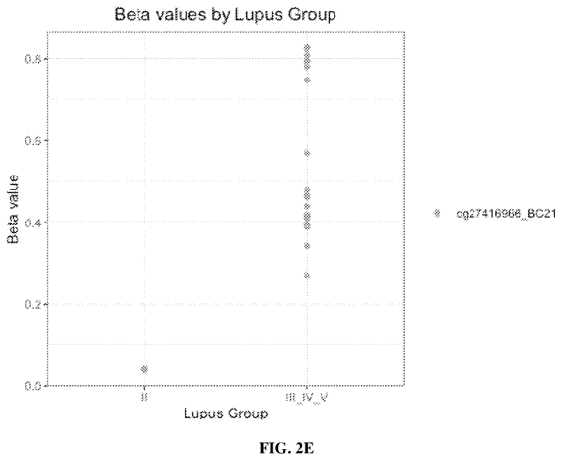

FIGS. 2A-2E are scatterplots demonstrating the beta values (percent methylation) of class II vs. III_IV_V at the top 5 differentially methylated CpG loci. Cg05108591_BC21 is hypomethylated in Class II relative to Class III, IV, and V; Cg09713586_BC21 is hypermethylated in Class II relative to Class III, IV, and V; Cg13350270_BC21 is hypomethylated in Class II relative to Class III, IV, and V′ Cg23059826_BC21 is hypomethylated in Class II relative to Class III, IV, and V; and Cg27416966_BC21 is hypomethylated in Class II relative to Class III, IV, and V.

Using KEGG enrichment pathway analysis, the top differentially methylated pathways between class II vs. class III/IV/V were identified. The pathways are presented below in Table 2 with their associated q-values.

TABLE 3
KEGG enrichment pathway analysis, the top differentially methylated
pathways between class II vs. class III/IV/V Lupus.

| Description | ID | qvalue |
|---|---|---|
| Rap1 signaling pathway | hsa04015 | 8.18724744685119E−10 |
| Focal adhesion | hsa04510 | 1.91206176622368E−07 |
| MAPK signaling pathway | hsa04010 | 1.91206176622368E−07 |
| Proteoglycans in cancer | hsa05205 | 3.2390533346214E−07 |
| Regulation of actin cytoskeleton | hsa04810 | 3.2390533346214E−07 |
| Axon guidance | hsa04360 | 3.2390533346214E−07 |
| Apelin signaling pathway | hsa04371 | 3.2390533346214E−07 |
| Yersinia infection | hsa05135 | 1.61600753752893E−06 |
| Small cell lung cancer | hsa05222 | 1.61600753752893E−06 |
| Endocrine resistance | hsa01522 | 1.61600753752893E−06 |
| Chronic myeloid leukemia | hsa05220 | 1.61600753752893E−06 |
| Non-small cell lung cancer | hsa05223 | 2.05963561777388E−06 |
| Phospholipase D signaling pathway | hsa04072 | 3.01113828310556E−06 |
| Cushing syndrome | hsa04934 | 4.32990464009283E−06 |
| Glioma | hsa05214 | 7.22708379264748E−06 |
| Endocytosis | hsa04144 | 7.46404323393299E−06 |
| Sphingolipid signaling pathway | hsa04071 | 1.06497643343141E−05 |
| Human cytomegalovirus infection | hsa05163 | 1.22577853590088E−05 |
| Cellular senescence | hsa04218 | 1.22577853590088E−05 |
| PI3K-Akt signaling pathway | hsa04151 | 1.22577853590088E−05 |
| Pancreatic cancer | hsa05212 | 1.22577853590088E−05 |
| Human T-cell leukemia virus 1 | hsa05166 | 1.22577853590088E−05 |
| infection | | |
| Ras signaling pathway | hsa04014 | 1.74284884364022E−05 |
| Human papillomavirus infection | hsa05165 | 2.13745727814061E−05 |
| Prostate cancer | hsa05215 | 2.90638499454393E−05 |
| Leukocyte transendothelial | hsa04670 | 2.90638499454393E−05 |
| migration | | |
| Hepatocellular carcinoma | hsa05225 | 3.13449579379774E−05 |
| EGFR tyrosine kinase inhibitor | hsa01521 | 3.14936736842152E−05 |
| resistance | | |
| Adherens junction | hsa04520 | 3.86430555943342E−05 |
| Salmonella infection | hsa05132 | 4.42236110204428E−05 |
| Glutamatergic synapse | hsa04724 | 4.42236110204428E−05 |
| Toxoplasmosis | hsa05145 | 4.60986488581334E−05 |
| Pathogenic Escherichia coli | hsa05130 | 4.98465393202223E−05 |
| infection | | |
| Bacterial invasion of epithelial cells | hsa05100 | 5.59385621593737E−05 |
| Fc gamma R-mediated | hsa04666 | 5.86184432107482E−05 |
| phagocytosis | | |
| ErbB signaling pathway | hsa04012 | 6.98658559408921E−05 |
| Endometrial cancer | hsa05213 | 7.21221079060655E−05 |
| Gastric acid secretion | hsa04971 | 7.31269873774295E−05 |
| Acute myeloid leukemia | hsa05221 | 7.35470137153721E−05 |
| FoxO signaling pathway | hsa04068 | 9.07829741366124E−05 |
| Bladder cancer | hsa05219 | 9.50580104481909E−05 |
| Colorectal cancer | hsa05210 | 0.00011647385577271 |
| Circadian entrainment | hsa04713 | 0.000124026333734978 |
| Platelet activation | hsa04611 | 0.000134414596728578 |
| Cholinergic synapse | hsa04725 | 0.000139263383360335 |
| Relaxin signaling pathway | hsa04926 | 0.00014301685338933 |
| Motor proteins | hsa04814 | 0.000185142516877158 |
| Long-term potentiation | hsa04720 | 0.000188640822146 |
| Autophagy - animal | hsa04140 | 0.000190409829338172 |
| cGMP-PKG signaling pathway | hsa04022 | 0.000190409829338172 |
| Renal cell carcinoma | hsa05211 | 0.000245808257892031 |
| Wnt signaling pathway | hsa04310 | 0.000245808257892031 |
| Gastric cancer | hsa05226 | 0.000343892771642898 |
| Growth hormone synthesis, | hsa04935 | 0.000355446632334883 |
| secretion and action | | |
| Shigellosis | hsa05131 | 0.000366109734404283 |
| Tight junction | hsa04530 | 0.000376913128193604 |
| Parathyroid hormone synthesis, | hsa04928 | 0.000409499399438855 |
| secretion and action | | |
| Central carbon metabolism in | hsa05230 | 0.000431051717721968 |
| cancer | | |
| Longevity regulating pathway | hsa04211 | 0.000499809856144719 |
| Thyroid hormone signaling | hsa04919 | 0.000509828210096391 |
| pathway | | |
| Oxytocin signaling pathway | hsa04921 | 0.000601720467649712 |
| Arrhythmogenic right ventricular | hsa05412 | 0.000656563067249074 |
| cardiomyopathy | | |
| Pathways of neurodegeneration - | hsa05022 | 0.000691869929750472 |
| multiple diseases | | |
| Hippo signaling pathway - multiple | hsa04392 | 0.000726016196003046 |
| species | | |
| Neurotrophin signaling pathway | hsa04722 | 0.000818610931958097 |
| cAMP signaling pathway | hsa04024 | 0.00081967672614661 |
| VEGF signaling pathway | hsa04370 | 0.000893748557515833 |
| Epstein-Barr virus infection | hsa05169 | 0.000929563022708612 |
| GABAergic synapse | hsa04727 | 0.000987063743449601 |
| Phosphatidylinositol signaling | hsa04070 | 0.000987063743449601 |
| system | | |
| Kaposi sarcoma-associated | hsa05167 | \|0.0010025078584414 |
| herpesvirus infection | | |
| mTOR signaling pathway | hsa04150 | 0.00108372088376876 |
| Insulin signaling pathway | hsa04910 | 0.00144165232327838 |
| Calcium signaling pathway | hsa04020 | 0.00154931042531091 |
| Adrenergic signaling in | hsa04261 | 0.00166526087073441 |
| cardiomyocytes | | |
| B cell receptor signaling pathway | hsa04662 | 0.00181692768778322 |
| Mucin type O-glycan biosynthesis | hsa00512 | 0.00195948432660366 |
| Spinocerebellar ataxia | hsa05017 | 0.00198833613706071 |
| Human immunodeficiency virus 1 | hsa05170 | 0.00214947903900082 |
| infection | | |
| Hedgehog signaling pathway | hsa04340 | 0.00217140212505084 |
| Morphine addiction | hsa05032 | 0.0022384712279441 |
| Chemokine signaling pathway | hsa04062 | 0.0022384712279441 |
| Viral carcinogenesis | hsa05203 | 0.00233352329369023 |
| Cell adhesion molecules | hsa04514 | 0.00234910125324281 |
| Melanoma | hsa05218 | 0.00239010686178949 |
| GnRH signaling pathway | hsa04912 | 0.00251685684981473 |
| Choline metabolism in cancer | hsa05231 | 0.00263518898996632 |
| Glycosphingolipid biosynthesis - | hsa00604 | 0.0027470935383102 |
| ganglio series | | |
| Apoptosis | hsa04210 | \|0.00276268417383709 |
| Lysine degradation | hsa00310 | 0.00296958813861773 |
| Purine metabolism | hsa00230 | 0.00299813219445136 |
| Fatty acid metabolism | hsa01212 | 0.00344937251907988 |
| Cortisol synthesis and secretion | hsa04927 | 0.00359013348263912 |
| Thyroid cancer | hsa05216 | 0.00359013348263912 |
| beta-Alanine metabolism | hsa00410 | 0.00359013348263912 |
| Breast cancer | hsa05224 | 0.00359940979314976 |
| HIF-1 signaling pathway | hsa04066 | 0.00382018763122515 |
| Gap junction | hsa04540 | 0.00425800539572571 |
| ABC transporters | hsa02010 | 0.004324225507622 |
| AMPK signaling pathway | hsa04152 | 0.00433727054095562 |
| Inflammatory mediator regulation | hsa04750 | 0.00440891422880596 |
| of TRP channels | | |
| Aldosterone synthesis and secretion | hsa04925 | 0.00440891422880596 |
| Mitophagy - animal | hsa04137 | 0.00440891422880596 |
| Osteoclast differentiation | hsa04380 | 0.00470319311807571 |
| Th1 and Th2 cell differentiation | hsa04658 | 0.00536905305727888 |
| Hippo signaling pathway | hsa04390 | 0.00536905305727888 |
| Dopaminergic synapse | hsa04728 | 0.00569182843020437 |
| Viral life cycle - HIV-1 | hsa03250 | 0.0057282630925047 |
| PD-L1 expression and PD-1 | hsa05235 | 0.00581789159341742 |
| checkpoint pathway in cancer | | |
| Vascular smooth muscle | hsa04270 | 0.00605545584507527 |
| contraction | | |
| C-type lectin receptor signaling | hsa04625 | 0.00605545584507527 |
| pathway | | |
| T cell receptor signaling pathway | hsa04660 | 0.00605545584507527 |
| Alzheimer disease | hsa05010 | 0.0062305529234362 |
| Inositol phosphate metabolism | hsa00562 | 0.00633955828564376 |
| Cocaine addiction | hsa05030 | 0.00633955828564376 |
| Melanogenesis | hsa04916 | 0.00650619560740241 |
| Propanoate metabolism | hsa00640 | 0.00650619560740241 |
| AGE-RAGE signaling pathway in | hsa04933 | 0.00819407446829973 |
| diabetic complications | | |
| Sphingolipid metabolism | hsa00600 | 0.00956996494090762 |
| Signaling pathways regulating | hsa04550 | 0.0110778448679703 |
| pluripotency of stem cells | | |
| Dilated cardiomyopathy | hsa05414 | 0.0113571368978322 |
| Other types of O-glycan | hsa00514 | 0.0113996498717573 |
| biosynthesis | | |
| Fc epsilon RI signaling pathway | hsa04664 | 0.0113996498717573 |
| p53 signaling pathway | hsa04115 | 0.0117366638051338 |
| Prolactin signaling pathway | hsa04917 | 0.0130658636289244 |
| Glycosaminoglycan biosynthesis - | hsa00532 | 0.0143176214830464 |
| chondroitin sulfate/dermatan | | |
| sulfate | | |
| Amoebiasis | hsa05146 | 0.0150835063470176 |
| Retrograde endocannabinoid | hsa04723 | 0.0160104629776776 |
| signaling | | |
| Amyotrophic lateral sclerosis | hsa05014 | 0.0161623299804247 |
| Adipocytokine signaling pathway | hsa04920 | 0.0163043733735008 |
| Ubiquinone and other terpenoid- | hsa00130 | 0.0164747644552529 |
| quinone biosynthesis | | |
| ECM-receptor interaction | hsa04512 | 0.0164853646691326 |
| Transcriptional misregulation in | hsa05202 | 0.0164853646691326 |
| cancer | | |
| Hepatitis B | hsa05161 | 0.0164853646691326 |
| Th17 cell differentiation | hsa04659 | 0.0190471556685972 |
| One carbon pool by folate | hsa00670 | 0.0202645991865039 |
| Long-term depression | hsa04730 | 0.0218617557149713 |
| Viral myocarditis | hsa05416 | 0.0218617557149713 |
| TNF signaling pathway | hsa04668 | 0.0240608097545914 |
| Glycerophospholipid metabolism | hsa00564 | 0.0259218205873484 |
| Ubiquitin mediated proteolysis | hsa04120 | 0.0273651879150062 |
| Homologous recombination | hsa03440 | 0.0273651879150062 |
| Ferroptosis | hsa04216 | 0.0273651879150062 |
| Amphetamine addiction | hsa05031 | 0.0275387185573842 |

Enrichment analysis revealed significant involvement of Rap1 signaling, focal adhesion, and MAPK signaling pathways (q-values <0.001). Hypermethylation of ERK and hypomethylation of RAP1 were identified in more active nephritis.

The results presented herein illustrate that there are differentially methylated CpG loci between patients with lupus nephritis class II vs. classes III, IV, and V. The Rap1 and MAPK signaling pathways, which are of importance in more active nephritis and have been implicated in the pathophysiology of SLE. All of the loci identified herein that were differentially methylated between participants with class II nephritis and those with III, IV, or V, and particularly the 5 CpG loci (adjusted p-values <0.05), could serve as potential biomarkers of lupus nephritis and help inform the decision to obtain kidney biopsy and subsequent treatment.

### Example 2: Blood DNA Methylation is Associated with Histopathological Changes in Lupus Nephritis

Approximately 60% of patients with systemic lupus erythematosus (SLE) develop nephritis. Histologically-determined disease classification provides clinically actionable information including administration of kidney-specific immunosuppression. Given the pivotal role of autoimmunity in SLE, experiments were designed to determine if DNA methylation patterns from immune cells would be associated with histopathological changes from kidney biopsy in individuals with lupus nephritis.

### Methods

28 participants were identified from the Yale Kidney Biobank with biopsy-confirmed lupus nephritis. DNA was extracted from blood buffy coat. DNA methylation was analyzed using Illumina MethylationEPIC V2.0, surveying >935,000 CpG loci. β-values (percent methylation) were derived using the R Sesame package. A single renal pathologist scored all kidney biopsies according to the NIH Activity and Chronicity Indices. The Bioconductor limma package was utilized to determine if there were differentially methylated loci by overall indices, as well as by each component of disease activity and chronicity. Benjamini-Hochberg method was used for false discovery rate correction.

### Results

The cohort was composed of participants with the following: class I (n=1), class II (n=3), class III (n=5), class III+V (n=3), class IV (n=4), class IV+V (n=1), class V (n=9), and class VI (n=2). The median activity index was 3 (IQR 1, 7). The median chronicity index was 3 (IQR 2, 6.25). In unadjusted analyses, differentially methylated loci (q<0.05) identified by scores including: fibrinoid necrosis, cellular/fibrocellular crescents, gobal glomerulosclerosis, fibrous crescents, and total chronicity index (Table 4). After adjustment for age, BMI, sex, and eGFR, loci remained significantly associated with fibrinoid necrosis, cellular/fibrocellular crescents and fibrous crescents (Table 4). 298 CpG loci associated with double-stranded DNA antibody level were also identified.

These findings indicate that DNA methylation patterns from immune cells correlate with certain histopathologic findings, particularly crescent formation, from kidney biopsy in patients with lupus nephritis.

TABLE 4
Differential methylation analyses by histopathological components of NIH

| | | | Analyses adjusted for age, | |
|---|---|---|---|---|
| | Unadjusted Analyses | | BMI, sex, eGFR | |
| | Number of | Number of | Number of | Number of |
| | CpG loci | CpG loci | CpG loci | CpG loci |
| | with q- | with p- | with q- | with p- |
| Biopsy parameter | value < 0.05 | value < 0.001 | value < 0.05 | value < 0.001 |
| Endocapillary | 0 | 496 | 0 | 335 |
| hypercellularity | | | | |
| Neutrophils/karyorrhexis | 0 | 310 | 0 | 280 |
| Fibrinoid necrosis | 882 | 3096 | 667 | 2376 |
| Hyaline deposits | 0 | 299 | 0 | 245 |
| Cellular/Fibrocellular | 3 | 599 | 5 | 744 |
| crescents | | | | |
| Interstitial inflammation | 0 | 969 | 0 | 2675 |
| Total NIH Activity Index | 0 | 340 | 0 | 453 |
| Global | 2 | 1175 | 0 | 426 |
| glomerulosclerosis | | | | |
| Fibrous crescents | 195 | 1344 | 176 | 1029 |
| Tubular atrophy | 0 | 760 | 0 | 1512 |
| Interstitial Fibrosis | 0 | 760 | 0 | 1512 |
| Total NIH Chronicity | 2 | 1149 | 0 | 759 |
| Index | | | | |

### Activity and Chronicity Indices.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific forms of the invention described herein. Such equivalents are intended to be encompassed by the following claims.

1. A method of detecting the methylation status at one or more locations in DNA from a human subject comprising processing DNA of a sample from the subject with a machine-based platform and detecting DNA methylation at and/or close to the genomic sequences associated with one or more CpG loci selected from the group consisting of cg09713586_BC21, cg23059826_TC21, cg13350270_BC21, cg27416966_BC21, cg05108591_BC21, cg04040637_BC21, cg22350135_BC21, cg26419274_TC21, cg12477512_BC21, cg17310601_BC21, cg19741112_TC21, cg11661493_TC21, cg10419144_TC21, cg25901241_BC21, cg08637101_BC21, cg12062757_BC21, cg16552271_TC21, cg00315056_TC21, cg03444505_BC21, cg06093355_BC21, cg04378177_TC21, cg16538390_BC21, cg15183647_BC21, cg04994762_BC21, cg13745646_BC21, cg18832251_BC21, cg20695119_BC21, cg02884024_BC11, cg06441554_BC21, cg04806722_BC21, cg22249302_BC21, cg07044006_BC21, cg05764585_TC21, cg16284456_TC21, cg14061946_BC21, cg09463032_TC11, cg02748908_BC11, cg24057166_TC21, cg13283765_BC21, cg13561626_BC21, cg07642801_BC21, cg11130441_TC21, cg06419236_TC21, cg09398527_TC21, cg10085612_BC21, cg06237697_TC11, cg17550329_BC21, cg26615813_TC11, cg11317269_TC21, and cg21288300_BC21.

2. The method of claim 1 comprising detecting DNA methylation at and/or close to the genomic sequences associated with one or more CpG loci selected from the group consisting of cg09713586_BC21, cg23059826_TC21, cg13350270_BC21, cg27416966_BC21, cg05108591_BC21, cg04040637_BC21, cg22350135_BC21, cg26419274_TC21, cg12477512_BC21, and cg17310601_BC21.

3. (canceled)

4. The method of claim 1 comprising detecting DNA methylation at and/or close to the genomic sequences associated with any integer number of the CpG loci between 2 and 50.

5. The method of claim 1, wherein the processing comprises one or more of PCR, digital droplet PCR, methylation specific PCR, real time methylation specific PCR, and PCR using a methylated DNA specific binding protein, quantitative PCR, quantitative real-time PCR, microarray, or DNA sequencing.

6. The method of claim 1, wherein the DNA is chemically-modified.

7. (canceled)

8. (canceled)

9. The method of claim 1, wherein sample is a blood sample, a urine sample, or kidney cells or tissue optionally a kidney biopsy.

10. A method of determining lupus nephritis class comprising detecting methylation at one or more differentially methylated CpG loci and comparing the level of methylation to one or more controls.

11. The method of claim 10, wherein the control(s) are known methylation values for the same CpG loci in subjects previously categorized as being Class II or less, or Class III or higher.

12. The method of claim 10, wherein the control(s) are known methylation values for the same CpG loci in subjects previously diagnosed as being Class II, Class III, Class IV, or Class V.

13. The method of claim 10, wherein methylation is detected according a method comprising processing DNA of a sample from the subject with a machine-based platform and detecting DNA methylation at and/or close to the genomic sequences associated with one or more CpG loci selected from the group consisting of cg09713586 BC21, cg23059826 TC21, cg13350270 BC21, cg27416966 BC21, cg05108591 BC21, cg04040637 BC21, cg22350135 BC21, cg26419274 TC21, cg12477512 BC21, cg17310601 BC21, cg19741112 TC21, cg11661493 TC21, cg10419144 TC21, cg25901241 BC21, cg08637101 BC21, cg12062757 BC21, cg16552271 TC21, cg00315056 TC21, cg03444505 BC21, cg06093355 BC21, cg04378177 TC21, cg16538390 BC21, cg15183647 BC21, cg04994762 BC21, cg13745646 BC21, cg18832251 BC21, cg20695119 BC21, cg02884024 BC11, cg06441554 BC21, cg04806722_BC21, cg22249302 BC21, cg07044006 BC21, cg05764585 TC21, cg16284456 TC21, cg14061946 BC21, cg09463032 TC11, cg02748908 BC11, cg24057166 TC21, cg13283765 BC21, cg13561626 BC21, cg07642801 BC21, cg11130441 TC21, cg06419236 TC21, cg09398527 TC21, cg10085612 BC21, cg06237697 TC11, cg17550329 BC21, cg26615813 TC11, cg11317269 TC21, and cg21288300 BC21.

14. The method of claim 10, comprising detecting methylation at one or more of cg09713586_BC21, cg23059826_TC21, cg13350270_BC21, cg27416966_BC21, and cg05108591_BC21, wherein Cg05108591_BC21 is hypomethylated in Class II relative to Class III, IV, and V; Cg09713586_BC21 is hypermethylated in Class II relative to Class III, IV, and V; Cg13350270_BC21 is hypomethylated in Class II relative to Class III, IV, and V′ Cg23059826_BC21 is hypomethylated in Class II relative to Class III, IV, and V; and Cg27416966_BC21 is hypomethylated in Class II relative to Class III, IV, and V.

15. The method of claim 1, comprising treating the subject for the class of lupus nephritis that is determined.

16. The method of claim 15, wherein subjects with Class II or less disease are not treated with an immunosuppressant and/or wherein subjects with Class III or higher disease are treated with an immunosuppressant.

17. (canceled)

18. The method of claim 15, wherein
(i) subjects with Class II or less are treated with one or more, optionally all of a renin-angiotensin-aldosterone system (RAAS) blocking agent, hydroxychloroquine, and a calcineurin inhibitor (CNI) or glucocorticoids (GC);
(ii) subjects with Class III or higher disease are treated with one or more, optionally all of, RAAS blockage, hydroxychloroquine, and additional immunosuppressive (IS) therapy: GC and either cyclophosphamide (CYC) or mycophenolate mofetil (MMF) or CNI or MMF and CNI;
(iii) subjects with Class IV or higher disease are treated with one or more, optionally all of, RAAS blockade, hydroxychloroquine, GC and either CNI or MMF or CYC or azathioprine (AZA); and/or
(iv) subjects with Class V or higher disease are treated with chronic kidney disease (CKD) management.

19.-21. (canceled)

22. A method of determining the likelihood of having one or more symptoms of lupus nephritis in a subject comprising detecting methylation at one or more CpG loci in DNA of a sample from the subject and comparing the level of methylation to one or more controls.

23. The method of claim 22, wherein the symptom is endocapillary hypercellularity, neutrophils/karyorrhexis, fibrinoid necrosis, hyaline deposits, cellular/fibrocellular crescents, interstitial inflammation, total NIH activity index, global glomerulosclerosis, fibrous crescents, tubular atrophy, interstitial fibrosis, total NIH chronicity index, or a combination thereof.

24. The method of claim 23, wherein the control(s) is methylation at one or more of the same CpG loci in a sample from a subject with lupus nephritis whose symptoms have been characterized by kidney biopsy, and/or
wherein a DNA methylation pattern in the subject that is the same or similar to the control(s) indicates the subject has one or more of the same symptoms as the control(s).

25. (canceled)

26. The method of claim 22, wherein the DNA sample is from blood, optionally the buffy coat, optionally immune cells therein.

27. The method of claim 22, wherein the detecting is according to a method comprising processing DNA of a sample from the subject with a machine-based platform and detecting DNA methylation at and/or close to the genomic sequences associated with one or more CpG loci selected from the group consisting of cg09713586 BC21, cg23059826 TC21, cg13350270 BC21, cg27416966 BC21, cg05108591 BC21, cg04040637 BC21, cg22350135 BC21, cg26419274 TC21, cg12477512 BC21, cg17310601 BC21, cg19741112 TC21, cg11661493 TC21, cg10419144 TC21, cg25901241 BC21, cg08637101 BC21, cg12062757 BC21, cg16552271 TC21, cg00315056 TC21, cg03444505 BC21, cg06093355 BC21, cg04378177 TC21, cg16538390 BC21, cg15183647 BC21, cg04994762 BC21, cg13745646 BC21, cg18832251 BC21, cg20695119 BC21, cg02884024 BC11, cg06441554 BC21, cg04806722_BC21, cg22249302 BC21, cg07044006 BC21, cg05764585 TC21, cg16284456 TC21, cg14061946 BC21, cg09463032 TC11, cg02748908 BC11, cg24057166 TC21, cg13283765 BC21, cg13561626 BC21, cg07642801 BC21, cg11130441 TC21, cg06419236 TC21, cg09398527 TC21, cg10085612 BC21, cg06237697 TC11, cg17550329_BC21, cg26615813_TC11, cg11317269_TC21, and cg21288300_BC21.

28. The method of claim 22, further comprising treating the subject for one or more of the symptoms.

29. (canceled)
